(19) United States
(12) Patent Application Publication
(10) Pub. No.: US 2024/0302345 A1
(43) Pub. Date: Sep. 12, 2024

(54) CHIP AGITATION DEVICE FOR A FLUID SYSTEM

(71) Applicant: Textron Innovations Inc., Providence, RI (US)

(72) Inventors: Colton Gilliland, Northlake, TX (US); Nathaniel Bernklau, Keller, TX (US)

(73) Assignee: Textron Innovations Inc., Providence, RI (US)

(21) Appl. No.: 18/179,021

(22) Filed: Mar. 6, 2023

(51) Int. Cl. G01N 33/28; B64C 27/00; B64F 5/60

(52) U.S. Cl. CPC G01N 33/2858; B64C 27/006; B64F 5/60; G01N 33/2888

(57) ABSTRACT

A chip agitation system for use on a rotorcraft with an engine and transmission system. The chip agitation system is configured to produce a mass flux of fluid to agitate chip debris towards a chip detection device. The chip agitation system includes a reservoir and valve that can be selectively opened and closed.

### BACKGROUND

Certain rotorcraft are capable of taking off, hovering, and landing vertically with little or no forward momentum. One such rotorcraft is a helicopter. Helicopters have one or more main rotors that generate a lifting force by rotating a plurality of rotor blades. The plurality of rotor blades is rotated by and structurally coupled to a rotor mast. The rotor mast is, in turn, powered and spun about a central axis by a main engine and a transmission and gearbox system. To function properly, the transmission and gearbox system must be constantly cooled and lubricated by a fluid system; but, even when properly cooled and lubricated, the transmission and gearbox system remain susceptible to spalling. Spalling occurs when the transmission and gearbox system deteriorates and breakdowns due to friction. If allowed to persist, spalling can lead to the mechanical failure of the transmission and gearbox system. Typically, spalling will generate chip debris, which flows into the fluid system. To avoid unnecessary damage to the transmission and gearbox system, it is advantageous to quickly detect this chip debris.

One means of detecting chip debris is a chip detection system. Generally, metal chips generated by spalling have a small mass and move with flowing fluid throughout the fluid system. The chip detection system is placed in the path of the flowing fluid, in such a manner that both the fluid and chip debris flow towards the chip detection system. The chip detection system will then filter for chip debris using a magnetic field, which attracts and catches metal chips. Upon contact with chip debris, the chip detection system electronically signals and warns maintenance personnel of potential spalling damage. Unfortunately, larger pieces of chip debris can be too heavy to move with flowing fluid; and, as a result, may not be detected by a traditional chip detection system. This can leave serious spalling damage undiagnosed.

### SUMMARY

In a first aspect, the present disclosure is directed to a fluid flow system for use in a mechanical system: The fluid flow system includes a fluid flow path configured to receive a fluid mass flow therethrough, wherein the fluid flow path is in fluid communication with a sump. The fluid flow system further includes a chip detector and a chip agitation unit that are both in fluid communication with the fluid flow path. The chip agitation unit includes a reservoir with an interior bottom surface and enclosing side walls for selectively receiving a reservoir fluid mass. The chip agitation unit further includes a valve in fluid communication with the reservoir, the valve having a closed position and an open position. While the valve is in the closed position, movement of the reservoir fluid mass from the reservoir to the sump is restricted, and the sump receives the first mass flow. While the valve is in the open position, movement of the reservoir fluid mass to the sump is less restricted than when in the open position, and the reservoir fluid mass partially combines with the first fluid mass flow to form a fluid mass flux that agitates a chip towards the chip detector.

In certain embodiments, the fluidic flow system includes a second fluid flow path configured to receive a second fluid mass flow therethrough. The second fluid flow path is in fluid communication with the chip transport assist unit. Specifically, the reservoir selectively receives a portion of the second fluid mass flow and the second fluid mass flow makes up a portion of the reservoir fluid mass. In other embodiments, the reservoir is pre-filled with a volume of fluid that makes up a portion of the reservoir fluid mass. In some embodiments, the fluid flow system is integrated into a transmission system for a rotorcraft including a main rotor gearbox. In certain embodiments, the chip transport assist unit is placed above the sump and the valve is mounted to the bottom surface of the reservoir, in such a manner that, in the open position, a gravitational force partially moves the reservoir fluid mass to the sump. In other embodiments, the first fluid flow path is at a downwards incline, in such a manner that a gravitational force partially moves the first fluid mass flow. In some embodiments, the valve switches between the closed position and the open position in response to an environmental parameter. The environmental parameter can be a measurement of the fluid volume within the reservoir. Additionally, the valve can be tunable to switch between the closed position and open position at variable frequencies. In certain embodiments, the valve includes a CPU capable of receiving an electronic signal, wherein the valve switches between the closed position and the open position in response to the CPU receiving the electronic signal. In other embodiments, the sump is a wet sump. The wet sump can further include a drain pump to partially move the chip towards the chip detector. In some embodiments, the fluid flow system includes a filter to capture the chip. In certain embodiments, the chip detection system uses a magnetic field.

In a second aspect, the present disclosure is directed towards a method for agitating a chip in a fluid lubrication system. A fluid flow path carrying a fluid mass flow is in fluid communication with a reservoir. The reservoir, with a bottom surface and enclosing side walls; is partially filled with a volume of fluid. In turn, the reservoir is partially drained and a portion of the volume of fluid from the reservoir combines with a portion of the fluid mass flow to create a fluid mass flux. The fluid mass flux agitates the chip within the fluid lubrication system.

In certain embodiments, the method can include agitating the chip towards a chip detector system. The chip detector system, in turn, detects the chip. In other embodiments, a valve with an open and closed position that is in fluidic communication with the reservoir, can be opened to partially drain the reservoir. The valve can be further mounted along the bottom surface of the reservoir, in such a manner that, upon opening the valve, a gravitational force aids in partially draining a portion of the volume of fluid from the reservoir. The valve can be tuned to open and close at variable frequencies. In an alternate embodiment, the valve can also be tuned to open and close in response to the volume of fluid within the reservoir reaching a certain threshold

### BRIEF DESCRIPTION OF THE DRAWINGS

For a more complete understanding of the features and advantages of the present disclosure, reference is now made to the detailed description along with the accompanying figures in which corresponding numerals in the different figures refer to corresponding parts and in which:

FIG. 1A-1C are schematic illustrations of a rotorcraft having an engine and transmission system in accordance with embodiments of the present disclosure;

FIG. 2A-2B are illustrations of a transmission system including a chip agitation system in accordance with embodiments of the present disclosure;

FIG. 3 is an illustration of a transmission system including a chip agitation system in accordance with an alternate embodiment of the present disclosure; and

FIG. 4 is an simplified schematic view of a closed loop transmission system including a chip agitation system in accordance with an alternate embodiment of the present disclosure.

### DETAILED DESCRIPTION

While the making and using of various embodiments of the present disclosure are discussed in detail below, it should be appreciated that the present disclosure provides many applicable inventive concepts, which can be embodied in a wide variety of specific contexts. The specific embodiments discussed herein are merely illustrative and do not limit the scope of the present disclosure. In the interest of clarity, not all features of an actual implementation can be described in the present disclosure. It will of course be appreciated that in the development of any such actual embodiment, numerous implementation-specific decisions must be made to achieve the developer's specific goals, such as compliance with system-related and business-related constraints, which will vary from one implementation to another. Moreover, it will be appreciated that such a development effort might be complex and time-consuming but would be a routine undertaking for those of ordinary skill in the art having the benefit of this disclosure.

In the specification, reference may be made to the spatial relationships between various components and to the spatial orientation of various aspects of components as the devices are depicted in the attached drawings. However, as will be recognized by those skilled in the art after a complete reading of the present disclosure, the devices, members, apparatuses, and the like described herein can be positioned in any desired orientation. Thus, the use of terms such as “above,” “below,” “upper,” “lower” or other like terms to describe a spatial relationship between various components or to describe the spatial orientation of aspects of such components should be understood to describe a relative relationship between the components or a spatial orientation of aspects of such components, respectively, as the device described herein can be oriented in any desired direction. In addition, as used herein, the term “coupled” can include direct or indirect coupling by any means, including moving and/or non-moving mechanical connections.

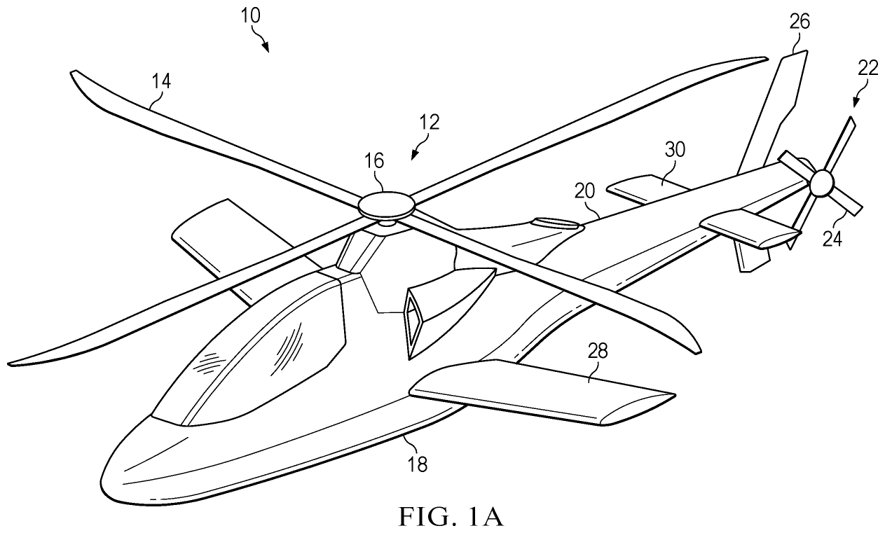

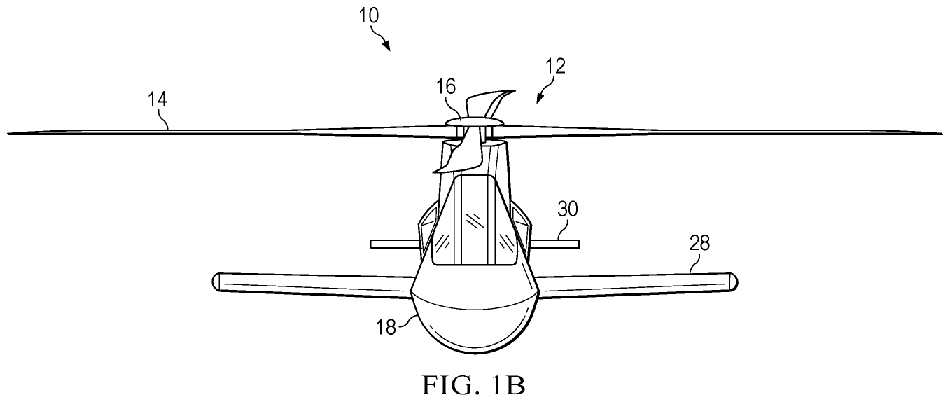

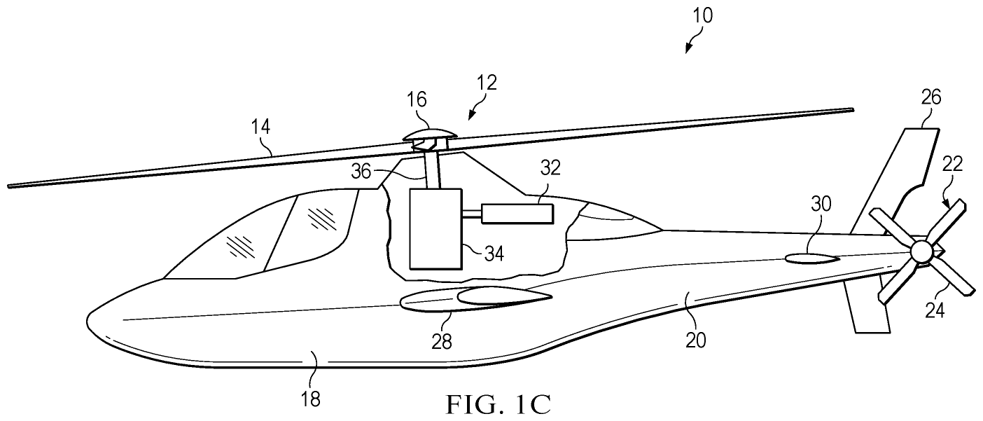

Referring to FIGS. 1A-1C in the drawings, a rotorcraft in the form of a helicopter is schematically illustrated and generally designated 10. The primary propulsion assembly of helicopter 10 is a main rotor assembly 12. Main rotor assembly 12 can include a plurality of rotor blades 14 extending radially outward from a main rotor hub 16. Main rotor assembly 12 is coupled to a fuselage 18 and is rotatable relative thereto. The pitch of rotor blades 14 can be collectively and/or cyclically manipulated to selectively control direction, thrust and lift of helicopter 10. A tailboom 20 is coupled to fuselage 18 and extends from fuselage 18 in the aft direction. An anti-torque system 22 can include a tail rotor assembly 24 coupled to an aft end of tailboom 20. Anti-torque system 22 controls the yaw of helicopter 10 by counteracting the torque exerted on fuselage 18 by main rotor assembly 12. In the illustrated embodiment, helicopter 10 can include a vertical tail fin 26 that provides stabilization to helicopter 10 during high-speed forward flight. In addition, helicopter 10 can include wing members 28 that extend laterally from fuselage 18 and horizontal stabilizers 30 that extend laterally from tailboom 20. Wing members 28 and horizontal stabilizers 30 provide lift to helicopter 10 responsive to the forward airspeed of helicopter 10, thereby reducing the lift requirement on main rotor assembly 12 and increasing the top speed of helicopter 10

Main rotor assembly 12 receives torque and rotational energy from a main engine 32 and a transmission system 34 within the fuselage 18. The main engine 32 can be coupled to the transmission system 34 by a clutching and shafting mechanism. Transmission system 34 can be, in turn, coupled to the main rotor assembly 12 by a rotor mast 36 mechanically coupled to the main rotor hub 16. Power from the main engine 32 is then used to spin the rotor mast 36. As the rotor mast 36 spins, it rotates the main rotor assembly 12.

It should be appreciated that helicopter 10 is merely illustrative of a variety of aircraft that can implement the embodiments disclosed herein. Other aircraft implementations can include hybrid aircraft, fixed wing aircraft, tiltwing aircraft, tiltrotor aircraft, quad tiltrotor aircraft, unmanned aircraft, gyrocopters, propeller-driven airplanes, compound helicopters, drones and the like. As such, those skilled in the art will recognize that the main rotor assembly and rotor mast disclosure can be integrated into a variety of aircraft configurations. It should be appreciated that even though aircraft are particularly well-suited to implement the embodiments of the present disclosure, non-aircraft vehicles and devices can also implement the embodiments.

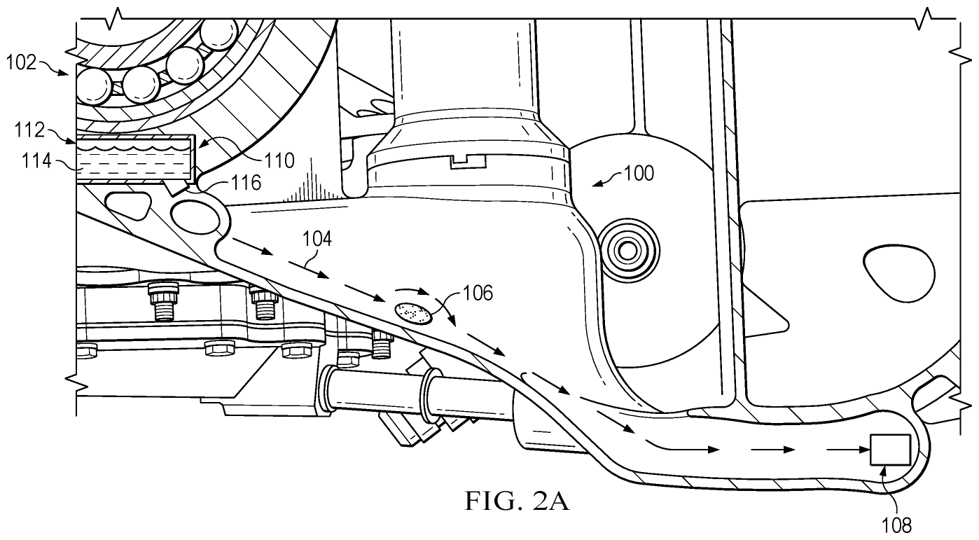

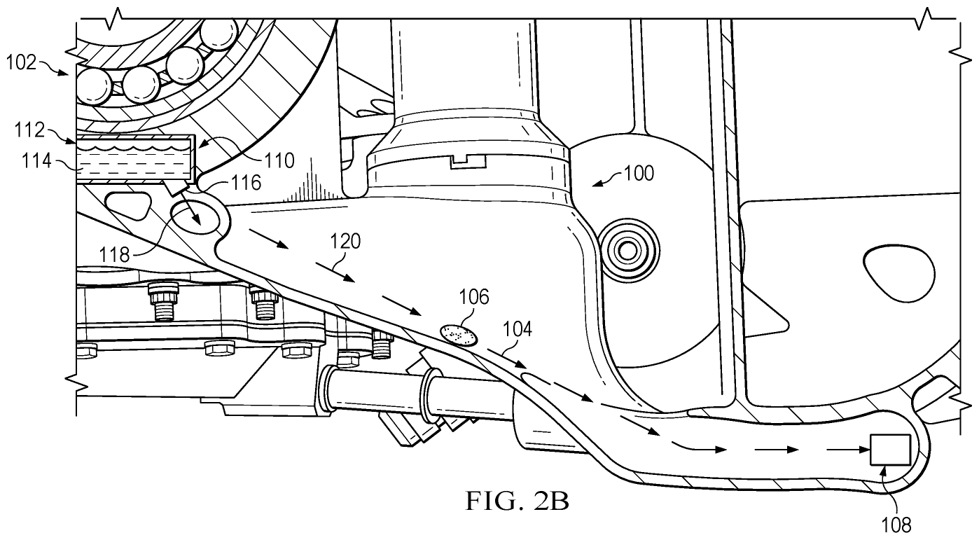

FIGS. 2A-2B are illustrations of a fluid system 100. The fluid system 100 comprises moving components including a gearbox and transmission system 102, which is susceptible to entropy and overheating. It should be noted that fluid system 100 is a dry sump system, wherein a majority of the fluid system's 100 volume is dry (i.e. not submerged in fluid). To lubricate and cool the moving components, gearbox, and transmission system 102, a fluid flow 104 is distributed throughout the fluid system 100. The fluid flow 104 in FIGS. 2A-2B moves downward with the assistance of gravity; but alternative embodiments of the fluid system 100 can propel fluid forward with vacuums, compressors, fluid pumps, or other means of fluid flow generation. During operation, friction causes spalling within the fluid system 100. Spalling, in turn, creates chip debris, which drops into the path of the fluid flow 104. A chip detection system 108 is placed within the flow path of the fluid flow 104, which, in turn, carries the chip debris towards the chip detection system 108. The chip detection system 108 then filters the chip debris from the fluid flow 104. Typically, the chip detection system 108 uses a magnetic field to separate chip debris from the fluid flow 104, but other means of filtration can be used as well. Unfortunately, the fluid flow 104 can not generate a sufficient force to move large-mass chip debris 106 toward the chip detection system 108. As a result, large-mass chip debris 106 can remain lodged in place and may not move towards the chip detection system 108, as best illustrated by FIG. 2A.

To agitate and move large-mass chip debris 106 towards the chip detection system 108, a chip agitation system 110 is integrated into the fluid system 100. The chip agitation system 110 includes a reservoir 112 and a valve 116 with a closed and an open position. The chip agitation system 110 is in fluid communication with the fluid flow 104, with a portion of the fluid flow 104 flowing into and partially filling the reservoir 112 with a volume of fluid 114. In alternative embodiments, the reservoir 112 can be pre-filled or partially filled by an alternative fluid source separate from fluid flow 104.

FIG. 2A shows valve 116 in the closed position. In the closed position, the valve 116 prevents fluid from exiting the reservoir 112, allowing a portion of the fluid flow 104 flowing into the reservoir 112 to fill the reservoir 112 with a volume of fluid 114. The volume of fluid 114 will continue to grow, until the reservoir 112 is full or the valve 116 switches into the open position.

FIG. 2B shows valve 116 in the open position. In the open position, at least a portion of the volume of fluid 114 exits the reservoir 112 through the valve 116, partially draining the volume of fluid 114 from the reservoir 112. Drained fluid 118 then flows out from the reservoir 112 into the fluid flow 104. It should be noted that the drained fluid 118 is propelled forward with the assistance of gravity; but, alternative embodiments of the invention can also use vacuums, compressors, fluid pumps, or other means of fluid flow generation to drain the reservoir 112. As the drained fluid 118 exits the reservoir 112, it combines with the fluid flow 104 to create a mass flux 120 of fluid. The mass flux 120, in turn, flows towards the large-mass chip debris 106 and chip detector 108. The mass flux's 120 flow force is greater than the fluid flow's 104 flow force; and, unlike the fluid flow's 104 flow force, the mass flux's 120 flow force can agitate and dislodge the large-mass chip debris 106 from its lodged position and push the large-mass chip debris 106 towards the chip detector 108. Once the mass flux 120 pushes the large mass chip debris 106 to the chip detector 108, the large-mass chip debris 106 can then be filtered from the fluid flow 104.

The valve 116 can either passively or actively switch between the open position and closed position. A passive valve system can switch the valve 116 between the opened and closed position in response to the volume of fluid 114 reaching a certain level of fluid within the reservoir 112. Alternatively, a passive valve system can open in response to weight, pressure, or temperature changes within the reservoir 112, or other environmental stimuli within the fluid system 100. Furthermore, a passive valve system can be tunable to open and close at variable or randomized frequencies, to better agitate the large-mass chip debris 106 with an inconsistent mass flux 120.

The valve 116, can also be configured to actively open and close in response to an electronic command signal from pilots, maintenance personnel, or other outside users. An active valve system can include a pneumatic, hydraulic, electronic, or other suitable actuation devices in mechanical communication with the valve 116. The actuation device can, in turn, be in electronic communication with a CPU capable of sending and receiving electronic signals. The actuation device can then actuate the valve 116 between and open and closed position in response to an electronic command signal received by the CPU. The CPU can then send a signal through a display or other suitable electronic device, informing pilots, maintenance personnel, or other outside users that the valve 116 is in the open or closed position.

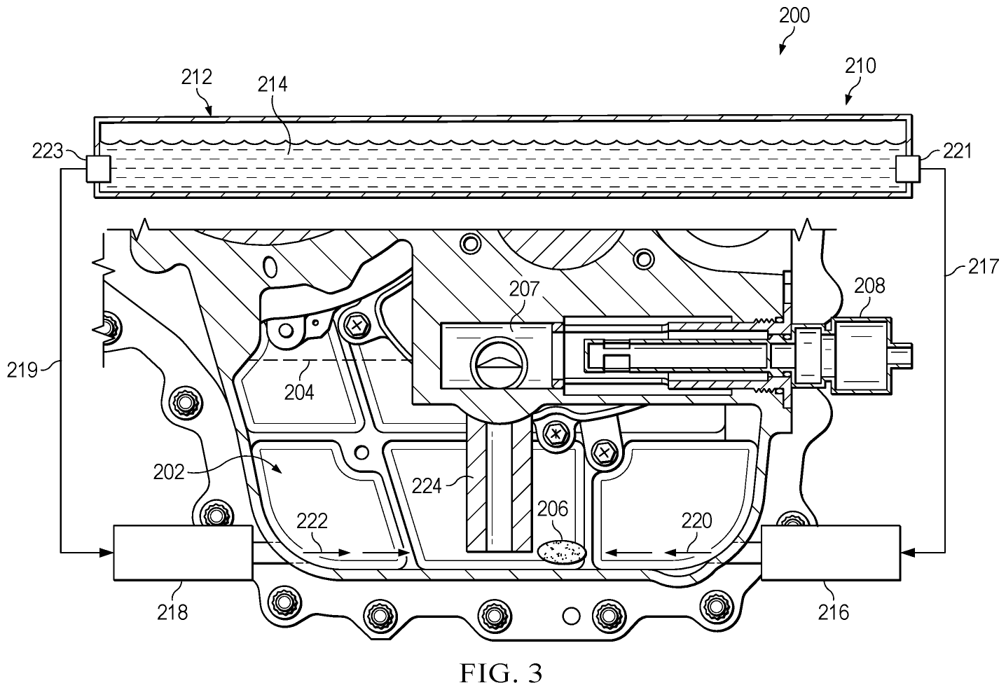

FIG. 3 is an illustration of a fluid system 200 in accordance with an alternative embodiment of the present disclosure. The fluid system 200 includes a wet sump reservoir 202, wherein the majority of the reservoir's volume is submerged in a fluid up to a fluid level line 204. Fluid continuously cycles into the wet sump reservoir 202 through a first and second transfer pump 216, 218. In turn, fluid continuously cycles out of the wet sump reservoir 202 through a drain pump 224. It should be noted that the two transfer pumps 216, 218 and one drain pump 224 are used to cycle fluid through the wet sump reservoir 202; however, alternate embodiments of the invention can use a different number and arrangement of transfer and drain pumps. Additionally, the first and second transfer pumps 216, 218 and drain pump 224 can be spatially situated in any number of suitable locations inside the wet sump reservoir 202 and are not limited to the spatial arrangement shown in FIG. 3.

Chip debris from spalling is generally submerged in the fluid and rests along the bottom of the wet sump reservoir 202. Drain pump 224 vacuums fluid and chip debris resting along the bottom of the wet sump reservoir 202 into an outlet 207. From the outlet 207, the fluid and chip debris flow towards a chip detector 208. Typically, the chip detector 208 uses a magnetic field to separate chip debris from the fluid in the outlet 207, but other means of filtration can also be used. Unfortunately, the drain pump 224 may not generate a sufficient force to vacuum large-mass chip debris 206 to the outlet 207. As a result, large-mass chip debris 206 can remain at the bottom of the first reservoir 204 and may not move towards the chip detection system 208.

To agitate and move large-mass chip debris 206 towards the chip detection system 208, a chip agitation system 210 is integrated into the fluid system 200. The chip agitation system 210 includes an agitation reservoir 212 filled with a volume of fluid 214 and a first and second reservoir valve 221, 223, wherein the first and second reservoir valves 221, 223 are in fluid communication with the agitation reservoir 212 and the first and second transfer pumps 216, 218 respectively. In the illustrated embodiments, the agitation reservoir 212 is pre-filled; but, in an alternate embodiment, can be partially or fully filled by an outside fluid source. The first and second reservoir valves 221, 223 have an open position and closed position. The first and second reservoir valves 221, 223 can either synchronously or independently switch between their respective open and closed positions. It should be noted that there are two reservoir valves 221, 223; however, alternate embodiments of the invention can use a different number and arrangement of reservoir valves.

In the closed position, the first and second reservoir valves 221, 223 prevent fluid from exiting the agitation reservoir 212.

FIG. 3 shows both the first and second reservoir valves 221, 223 in the open position. In the open position, at least a portion of the volume of fluid 214 exits the agitation reservoir 212 through the first and second reservoir valves 221,223, partially draining the volume of fluid 214 from the agitation reservoir 212. Drained fluid then flows out from the first and second reservoir valves 221, 223 through a fluid flow path 217, 219. It should be noted that the drained fluid is propelled forward with the assistance of gravity; but, alternative embodiments of the invention can also use vacuums, compressors, fluid pumps, or other means of fluid flow generation to drain the agitation reservoir 212. As the drained fluid exits the agitation reservoir 212, it combines with the fluid pumping through the transfer pumps 216, 218 to create respective mass fluxes 220,222 of fluid. The mass fluxes 220, 222, in turn, agitate the fluid within the wet sump reservoir 202. The resulting flow force from the mass fluxes 220, 222 can agitate and dislodge the large-mass chip debris 206 from its lodged position and push the large-mass chip debris 206 towards the drain pump 224. In turn, the drain pump 224 can carry the large-mass chip debris 206 to the chip detector 208. Once the drain pump 224 carries the large mass chip debris 206 to the chip detector 208, the large-mass chip debris 206 can then be filtered from the fluid.

The reservoir valves 221, 223 can either passively or actively switch between their respective open and closed positions. A passive valve system can switch the reservoir valves 221, 223 between the opened and closed position in response to the volume of fluid 214 within the reservoir 212. Alternatively, a passive valve system can open in response to weight, pressure, or temperature changes within the reservoir 212, or other environmental stimuli within the fluid system 200. Furthermore, a passive valve system can be tunable to open and close at variable or randomized frequencies, to better agitate the large-mass chip debris 206 with inconsistent mass fluxes 220, 222.

The reservoir valves 221, 223 can also be configured to actively open and close in response to an electronic command signal from pilots, maintenance personnel, or other outside users. An active valve system can include a pneumatic, hydraulic, electronic, or other suitable actuation devices in mechanical communication with the reservoir valves 221, 223. The actuation device can, in turn, be in electronic communication with a CPU capable of sending and receiving electronic signals. The actuation device can then actuate the reservoir valves 221, 223 between and open and closed position in response to an electronic command signal received by the CPU. The CPU can then send a signal through a display or other suitable communication device, informing pilots, maintenance personnel, or other outside users that the reservoir valves 221, 233 are in the open or closed positions. It should be noted that an active valve system can be configured to open valves 221, 223 either independently or synchronously with one another.

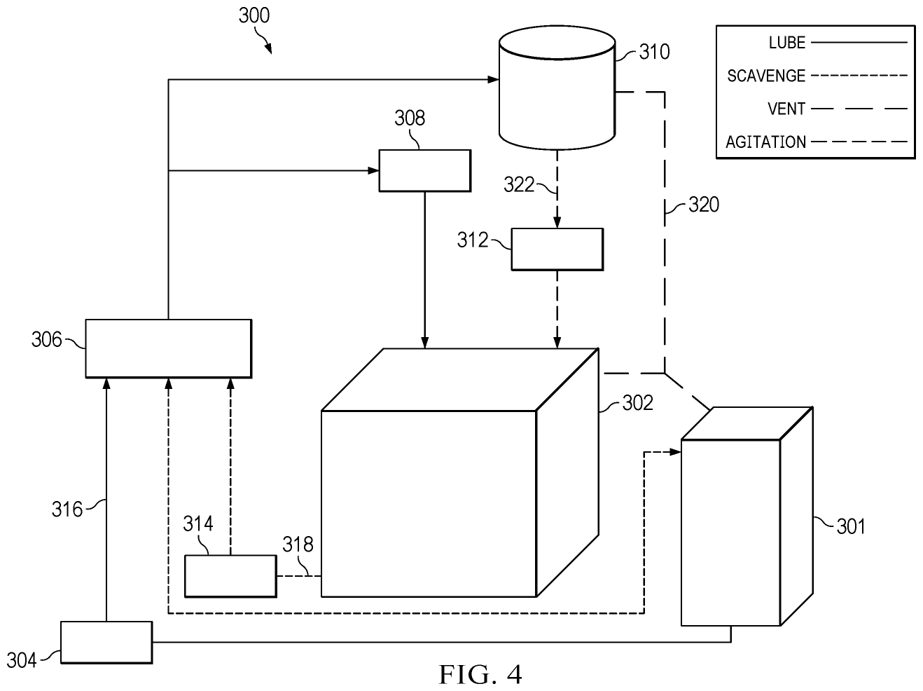

FIG. 4 shows a simplified schematic view of a closed loop fluid system 300. The closed loop fluid system 300 includes a wet sump system 301, wherein the majority of the wet sump system's 301 volume is submerged in fluid. Fluid exits the wet sump system 301 through a lubrication fluid line 316. The fluid from the lubrication line 316 then enters a heat exchanger 304, where the fluid is cooled. The cooled fluid from the lubrication line 316 is then propelled forward with a lubrication pump 306, wherein a portion of the cooled fluid is used as fluid jets 308 for a dry sump system 302 and another portion of the cooled fluid is used to fill the volume of a chip agitation reservoir 310. The cooled fluid from the fluid jets 308 then enters, cools, and lubricates the dry sump system 302. Residual fluid then exits the dry sump system 302 through a scavenge line 318. Fluid from the scavenge line 318 then enters a chip detector 314, wherein the fluid is screened and filtered for chip debris. The screened fluid is then propelled forward with the lubrication pump 306 and recycled through the wet sump reservoir 301. It should be noted that the lubrication pump 306 is used to propel fluid through both the lubrication line 316 and the scavenge line 318; however, in an alternate embodiment, separate pumps can be used for each fluid line.

In response to external or environmental stimuli, a chip agitation valve 312 can be switched between a closed position and an open position. In the closed position, cooled fluid collected from the lubrication line 316 remains in the chip agitation reservoir 310. In the open position, cooled fluid flows from the chip agitation reservoir 310 through an agitation line 322 to the chip agitation valve 312. From the chip agitation valve 312, fluid then flows through the agitation line 322 to the dry sump system 302. At the dry sump system 302, the fluid flowing through the agitation line 322 can combine with the fluid flowing through the lubrication line 316 to create a mass flux of fluid that is capable of agitating and dislodging large mass chip debris. Large mass chip debris, in turn, can then be propelled towards the chip detector 314 and filtered from the scavenge line 318. It should be appreciated that pressure between the chip agitation reservoir 310, wet sump system 301, and dry sump system 302 can be moderated and equalized using a vent line 320.

At least one embodiment is disclosed, and variations, combinations, and/or modifications of the embodiment(s) and/or features of the embodiment(s) made by a person having ordinary skill in the art are within the scope of this disclosure. Alternative embodiments that result from combining, integrating, and/or omitting features of the embodiment(s) are also within the scope of this disclosure. Where numerical ranges or limitations are expressly stated, such express ranges or limitations should be understood to include iterative ranges or limitations of like magnitude falling within the expressly stated ranges or limitations (e.g., from about 1 to about 10 includes, 2, 3, 4, etc.; greater than 0.10 includes 0.11, 0.12, 0.13, etc.). For example, whenever a numerical range with a lower limit, Rl, and an upper limit, Ru, is disclosed, any number falling within the range is specifically disclosed. In particular, the following numbers within the range are specifically disclosed: R=Rl+k*(Ru−Rl), wherein k is a variable ranging from 1 percent to 100 percent with a 1 percent increment, i.e., k is 1 percent, 2 percent, 3 percent, 4 percent, 5 percent, . . . 50 percent, 51 percent, 52 percent, . . . , 95 percent, 96 percent, 95 percent, 98 percent, 99 percent, or 100 percent. Moreover, any numerical range defined by two R numbers as defined in the above is also specifically disclosed.

Use of the term “optionally” with respect to any element of a claim means that the element is required, or alternatively, the element is not required, both alternatives being within the scope of the claim. Use of broader terms such as comprises, includes, and having should be understood to provide support for narrower terms such as consisting of, consisting essentially of, and comprised substantially of. Accordingly, the scope of protection is not limited by the description set out above but is defined by the claims that follow, that scope including all equivalents of the subject matter of the claims. Each and every claim is incorporated as further disclosure into the specification and the claims are embodiment(s) of the present invention. Also, the phrases “at least one of A, B, and C” and “A and/or B and/or C” should each be interpreted to include only A, only B, only C, or any combination of A, B, and C.

What is claimed is:

1. A fluid flow system for use in a mechanical system, comprising:
a first fluid flow path configured to receive a first fluid mass flow therethrough, wherein the first fluid flow path is in fluid communication with a sump, the sump comprising a chip detector in fluid communication with the first fluid flow path; and
a chip agitation unit in fluid communication with the sump, the chip agitation unit comprising:
a reservoir comprising an interior bottom surface and enclosing side walls for selectively receiving a reservoir fluid mass;
a valve in fluid communication with the reservoir, the valve operable between a closed position and an open position;
wherein, when the chip agitation unit is operated with the valve in the closed position, movement of the reservoir fluid mass from the reservoir to the sump is restricted, and the sump receives the first mass flow; and
wherein, when the chip agitation unit is operated with the valve in the open position, movement of the reservoir fluid mass to the sump is relatively less restricted than when in the valve is in the closed position, thereby allowing a portion of the reservoir fluid mass to combine with a portion of the first fluid mass flow to form a fluid mass flux for agitating a chip towards the chip detector.

2. The fluid flow system of claim 1 further comprising:
a second fluid flow path configured to receive a second fluid mass flow therethrough;
wherein the second fluid flow path is in fluid communication with the chip agitation unit; wherein the reservoir selectively receives the second fluid mass flow; and
wherein the second fluid mass flow partially comprises the reservoir fluid mass.

3. The fluid flow system of claim 1 further comprising a pre-filled volume of fluid that partially comprises the reservoir fluid mass.

4. The fluid flow system of claim 1, wherein the mechanical system comprises a transmission system for a rotorcraft comprising a main rotor gearbox.

5. The fluid flow system of claim 1, wherein the chip agitation unit is disposed vertically higher than the sump and the valve is mounted to the bottom surface of the reservoir, in such a manner that, when the valve is in the open position, a gravitational force moves the reservoir fluid mass to the sump.

6. The fluid flow system claim 1, wherein the first fluid flow path comprises a vertical decline, in such a manner that a gravitational force moves the first fluid mass flow.

7. The fluid flow system of claim 1, wherein the valve switches between the closed position and the open position in response to an environmental parameter.

8. The fluid flow system of claim 7, wherein the environmental parameter comprises a measurement of fluid volume within the reservoir.

9. The fluid flow system of claim 1, wherein the valve is tunable to switch between the closed position and the open position at variable frequencies.

10. The fluid flow system of claim 1, wherein the valve comprises a CPU capable of receiving an electronic signal and wherein the valve switches between the closed position and the open position in response to the CPU receiving the electronic signal.

11. The fluid flow system of claim 1, wherein the sump comprises a wet sump.

12. The fluid flow system of claim 1, further comprising:
a drain pump configured to partially move the chip towards the chip detector.

13. The fluid flow system of claim 1, further comprising:
a filter configured to capture the chip.

14. The fluid flow system of claim 1, wherein the chip detection system is configured to use a magnetic field.

15. A method for agitating a chip in a fluid lubrication system, the method comprising:
providing a fluid flow path configured to receive a fluid mass flow;
providing a reservoir comprising a bottom surface and enclosing side walls;
at least partially filling the reservoir with a volume of fluid;
at least partially draining the volume of fluid from the reservoir;
combining a portion of the volume of fluid with a portion of the fluid mass flow to create a fluid mass flux; and
agitating the chip with the fluid mass flux.

16. The method of claim 15, further comprising:
providing a chip detector system;
agitating the chip towards the chip detector system with the mass flux; and
detecting the chip with the chip detector.

17. The method of claim 16, further comprising:
providing a valve in fluidic communication with the reservoir, wherein the valve is configured to be selectively operated between an open position and a closed position; and
selectively opening the valve to at least partially drain the volume of fluid to combine with the fluid mass flow.

18. The method of claim 17, further comprising:
mounting the valve along the bottom surface of the reservoir, in such a manner that, upon at least partially opening the valve, a gravitational force aids in at least partially draining the volume of fluid from the reservoir.

19. The method of claim 18, further comprising:
changing a frequency at which the valve is operated between the open position and the closed position.

20. The method of claim 18 further comprising:
controlling the valve in response to a measurement of fluid volume within the reservoir.
